(19) United States
(12) Patent Application Publication
(10) Pub. No.: US 2010/0324137 A1
(43) Pub. Date: Dec. 23, 2010

(54) LAURIC ARGINATE AS A CONTACT ANTIMICROBIAL

(75) Inventor: Michael F. Coughlin, Cincinnati, OH (US)

Correspondence Address: Diversey, Inc., 8310 16TH STREET, M/S 509, PO BOX 902, STURTEVANT, WI, 53177-0902, US

(73) Assignee: DIVERSEY, INC., Sturtevant, WI (US)

(21) Appl. No.: 12/817,940

(22) Filed: Jun. 17, 2010

Related U.S. Application Data
(60) Provisional application No. 61/219,285, filed on Jun. 22, 2009.

(51) Int. Cl. A61K 31/221

(52) U.S. Cl. 514/551

(57) ABSTRACT

A surface disinfecting composition includes a combination of lauric arginate ethyl ester in combination with EDTA, octenyl succinic acid, octanoic acid, glycolic acid, lactic acid, or combinations thereof. The aqueous solution can be used to disinfect a wide variety of surfaces including food processing and storage equipment, vegetables and fruits, as well as animal carcasses such as poultry.

### BACKGROUND OF THE INVENTION

Ethyl lauroyl arginate is founed by esterfying arginine with ethanol, and subsequently reacting the ester with lauroyl chloride. The resultant ethyl lauroyl arginate is recovered as a hydrochloride salt. This is also referred to as lauric arginate ethyl ester, or simply lauric arginate, and is commonly referred to by the term LAE.

Lauric arginate ethyl ester has been promoted as a food preservative. It has also been used to a limited extent as a contact antimicrobial. However, it is basically cost prohibitive to use this as a contact antimicrobial for most applications.

### SUMMARY OF THE INVENTION

The present invention is premised on the realization that the antimicrobial effect of lauric arginate can be enhanced by the addition of one of more compounds, including ethylene diamine tetraacetic acid (EDTA), octenyl succinic acid, octanoic acid, glycolic acid, lactic acid, citric acid, levulinic acid, or mixtures thereof. By combining one or more of the above components with lauric arginate, the antimicrobial effect of the compound is enhanced, thereby reducing the amount of LAE required, and thus its cost.

In particular, this composition can be used as a contact antimicrobial, in particular to kill bacteria on the surfaces of fruits and vegetables, as well as on the surfaces of animal carcasses. Further, it can be incorporated into a surface cleaning composition.

The objects and advantages of the present invention will be further appreciated in light of the following detailed description.

### DETAILED DESCRIPTION

According to the present invention, ethyl lauroyl arginate, or lauric arginate ethyl ester (LAE) is combined with a second component to provide a disinfecting solution. The disinfecting solution is as an aqueous solution which is applied to a surface to disinfect the surface.

The disinfecting solution of the present invention will include LAE and a second component which is one or more of ethylene diamine tetracetic acid (EDTA), octenyl succinic acid, octanoic acid, glycolic acid, lactic acid, citric acid, levulinic acid, or mixtures thereof.

The LAE is commercially available as a water soluble powder and a 10-20% solution stabilized in glycol. The LAE is combined with one of the second components, and dissolved in water to form the disinfecting solution of the present invention. The ratio of the LAE to the second component will vary depending upon the particular second component, and further may vary depending on whether there will be two or more of the second components. However, in general the ratio of LAE to the second component will vary from 1:10 to 10:1 by weight.

Generally, in use, the concentration of LAE will be 5 ppm up to about 50 ppm. With respect to the EDTA, when it is used with respect to the LAE, the use concentration will be 10 ppm to 100 ppm. The use concentration of octenyl succinic acid, when combined with LAE, will be 20 to 200 ppm. The octanoic acid, when used, should be present in a range of 5 to 50 ppm. Likewise, with the glycolic, lactic, citric and levulinic acid, the use concentration should be 40 to 400 ppm.

The composition is formed by simply dissolving the LAE and the second component, or second components, in water.

The pH can be adjusted by adding either additional acid, particularly one of the acids listed as a second component, or by adding a base to increase the pH, such as sodium hydroxide. The pH may be adjusted to meet intended end use requirements. However, generally the pH will be acidic.

In addition to the LAE and second component, the disinfecting solution may include wetting agents such as the Tween surfactants, particularly Tween 80. Defoamers can also be added, if necessary. Again, these components would be added to meet end use requirements.

When EDTA is added to the lauric arginate, it is preferable to have additional EDTA to act as a sequestering agent or to blend the lauric arginate and EDTA in soft water. The EDTA naturally sequesters hardness ions. Therefore, if the hardness ions are present the EDTA will sequester the hardness ions and not be available for activity in combination with the lauric arginate. Other hardness sequestering compositions such as NTA can be used in combination with the lauric arginate and EDTA. Preferably, the lauric arginate can be combined with the EDTA in soft water to permit all the EDTA to work actively in combination with the lauric arginate.

The solution of the present invention is simply applied to a surface that is intended to be disinfected by any well known manner such as spraying, immersion, or the like. It can be used for a wide variety of applications. In particular, the present invention can be used to disinfect the exterior surface of vegetables, as well as the exterior surface of carcasses.

Application time can vary from a few seconds to several minutes before the antimicrobial is rinsed off the surface. To disinfect carcasses, the disinfecting solution is generally applied for 10-45 seconds.

The temperature of application can be any temperature above freezing and below the boiling point of the disinfectant composition. Generally, room temperature (22° C.) is adequate.

The invention will be further appreciated in light of the following detailed example.

### EXAMPLE 1

The antimicrobial effectiveness of the present invention in combination with octenyl succinic acid was tested. In this application, the test organism was E. coli, which was contacted with either the control or the stated sample for 30 seconds, and the survivors were calculated. The results are shown in Table 1.

TABLE 1

| Test | Sample Identification | Survivors | Percent |
|---|---|---|---|
| No. | (Concentrations in ppm Active) | (org/ml) | Reduction |
| A | Control | 24,800,000 | 83.896 |
| | | 26,400,000 | 82.857 |
| B | 160 ppm OSA | 24,100,000 | 84.351 |
| | | 23,400,000 | 84.805 |
| C | 320 ppm OSA | 2,200 | 99.999 |
| | | 2,000 | 99.999 |
| D | 480 ppm OSA | 1,900 | 99.999 |
| | | 1,920 | 99.999 |
| E | 25 ppm LA | 5,400,000 | 96.494 |
| | | 5,700,000 | 96.299 |
| F | 160 ppm OSA + 25 ppm LA | 1,800 | 99.999 |
| | | 1,600 | 99.999 |
| G | 320 ppm )SA + 25 ppm LA | 1,300 | 99.999 |
| | | 1,300 | 99.999 |
| H | 480 ppm OSA + 25 ppm LA | 2,000 | 99.999 |
| | | 2,000 | 99.999 |
| I | 50 ppm LA | 229,000 | 99.851 |
| | | 114,000 | 99.926 |
| J | 160 ppm OSA + 50 ppm LA | 2,000 | 99.999 |
| | | 1,900 | 99.999 |
| K | 320 ppm OSA + 50 ppm LA | 1,900 | 99.999 |
| | | 2,000 | 99.999 |
| L | 480 ppm OSA + 50 ppm LA | 2,300 | 99.999 |
| | | 2,500 | 99.998 |
| M | 75 ppm LA | 33,000 | 99.979 |
| | | 39,000 | 99.975 |
| N | 160 ppm OSA + 75 ppm LA | 1,800 | 99.999 |
| | | 1,600 | 99.999 |

As shown, the addition of octenyl succinic acid significantly enhanced the activity of the lauric arginate ethyl ester, and, further, the combination of lauric arginate with octenyl succinic acid surpassed the activity of octenyl succinic acid by itself. This demonstrates that by adding the octenyl succinic acid to the lauric arginate significantly improves efficacy thereby permitting a lower concentration of lauric arginate to be employed.

This has been a description of the present invention along with the preferred method of practicing the present invention. However, the invention itself should only be defined by the appended claims.

What is claimed is:

1. A composition for use in disinfecting surfaces comprising lauric arginate ethyl ester in combination with a second component wherein said second component is selected from the group consisting of EDTA, octenyl succinic acid, octanoic acid, glycolic acid, lactic acid, citric acid, levulinic acid, and combinations thereof.

2. The cleaning solution claimed in claim 1 wherein said solution has a concentration of lauric arginate ethyl ester of at least about 5 ppm.

3. The composition claimed in claim 2 wherein said second component is EDTA having a concentration of at least 10 ppm.

4. The composition claimed in claim 2 wherein said second component is octenyl succinic acid having a concentration of at least about 20 ppm.

5. The composition claimed in claim 2 wherein said second component is octanoic acid having a concentration of at least about 5 ppm.

6. The composition claimed in claim 2 wherein said second component is glycolic acid having a concentration of at least about 40 ppm.

7. The composition claimed in claim 2 wherein the second component is selected from the group consisting of lactic acid citric acid, levulinic acid, and combinations thereof, at a concentration of at least 40 ppm.

8. A method of disinfecting a surface comprising applying to said surface a composition comprising lauric arginate ethyl ester in combination with a second component selected from the group consisting of EDTA, octenyl succinic acid, octanoic acid, glycolic acid, lactic acid, citric acid, levulinic acid, and combinations thereof.

9. The method claimed in claim 8 wherein said surface comprises the surface of fruit or vegetables.

10. The method claimed in claim 8 wherein said surface comprises the surface of an animal carcass.
